(19) United States
(12) Patent Application Publication
(10) Pub. No.: US 2024/0360394 A1
(43) Pub. Date: Oct. 31, 2024

(54) MICROFLUIDIC CHIP SYSTEM FOR AUTOMATIC SEPARATION OF LIVE SPERM AND SUBSEQUENT FORMATION OF SINGLE-SPERM-ENCAPSULATED MICROHYDROGELS

(71) Applicants: City University of Hong Kong, Hong Kong (HK); Huazhong University of Science and Technology, Wuhan City (CN)

(72) Inventors: Xianjin XIAO, Wuhan (CN); Mengsu YANG, Hong Kong (HK); Xiaoyu ZHOU, Hong Kong (HK); Yaoqin MU, Wuhan (CN); Yuan WANG, Hong Kong (HK)

(21) Appl. No.: 18/646,757

(22) Filed: Apr. 26, 2024

Related U.S. Application Data
(60) Provisional application No. 63/498,524, filed on Apr. 27, 2023.

(51) Int. Cl. C12M 3/06; C12M 1/12; C12M 1/26; C12M 1/36; C12M 3/00; C12N 5/076

(52) U.S. Cl. CPC C12M 23/16; C12M 21/06; C12M 25/16; C12M 33/04; C12M 41/48; C12N 5/061; C12N 2533/74

(57) ABSTRACT

The present invention relates to a Biochemical-Level Automatic-screening Smart droplet-TO-micro-hydrogels chip (BLASTO-chip) system for sperm selection. The BLASTO-chip technology advances sperm selection from the primitive morphology level to a more sophisticated biochemical level, and it will not only provide a powerful tool to patients who have fertility problems but also work as a platform for further development of more advanced sperm selection technologies. The present invention also provides a method for improving the success rate of in vitro fertilization in a patient with asthenospermia.

### CROSS-REFERENCE TO RELATED APPLICATIONS

The present application claims the priorities from the U.S. provisional patent application Ser. No. 63/498,524 filed Apr. 27, 2023, and the disclosure of which is incorporated herein by reference in its entirety.

### FIELD OF THE INVENTION

The present invention generally relates to at least the fields of reproductive medicine, embryology, etc. more specifically, the present invention relates to a BLASTO-chip system for automatic high-throughput and non-invasive selection of single sperm at biochemical level.

### BACKGROUND OF THE INVENTION

Infertility is a growing global health problem that affects up to 186 million people. About 30% of these infertility cases are caused by male factors alone, while a combination of male and female factors causes nearly 40% of infertility cases. All infertility therapies using assisted reproductive technologies (ARTs), such as intrauterine insemination (IUI), in vitro fertilization (IVF) and intracytoplasmic monosperm injection (ICSI), call for the selection of high-quality sperm from semen samples to increase the success rate of insemination. Therefore, the selection of sperm is a highly critical step of ART.

Currently, the most commonly used sperm selection techniques (e.g., sperm morphology observation, swim-up method, density gradient centrifugation) are based on physical characteristics, such as sperm morphology and motility. Sperm with more standardized morphology and better motility are selected for further fertilization. However, it is evident that selection based on physical features cannot reflect sperm' biochemical status, which is essential for subsequent fertilization and blastocyst development as they are a series of complicated biochemical events.

In recent years, microfluidic based sperm selection was widely investigated. Most of the microfluidic devices select sperm by designing complex and twisted pathways so that only sperm with best motility could reach the endpoint. Obviously, the essence of those devices was still motility-based and had no difference with conventional selection methods. Later on, more complicated selection criteria were adopted in microfluidic devices, such as chemotaxis, thermotaxis and rheotaxis. However, those taxes were all based on the prerequisite of sperm's good motility. No matter conventional or later microfluidic sperm selection methods were employed, all of them faced limitations at the primitive physical level (morphology or motility). Consequently, sperm with good morphology and motility might still show low potential in the biochemical processes of fertilization and blastocyst development. Such “irrelevance” poses extremely tough problems for patients with severe and total asthenozoospermia: their sperm are almost immobile; therefore, conventional techniques cannot select sperm by motility and more troublesome, the techniques even cannot identify which sperm is alive. Under such circumstances, embryologists have to “blindly select” live sperm only by morphology for subsequent fertilization, and expectedly, the fertilization rate for such “blind selection” was only 10%-20%, which is much lower than the fertilization rate of normal sperm samples (approximately 80%).

In order to improve the ART outcome of such patients, several “invasive” techniques that can further assess the viability of sperm have been adopted. Examples include the hypo-osmotic swelling test, mechanical touch technique, exposure to pentoxifylline and laser-assisted immotile sperm selection. However, those invasive techniques not only present low efficiency in discriminating live and dead sperm but also directly alter the sperm's intracellular status, and such alteration might be inherited by offspring and cause immeasurable repercussions.

The fundamental cause of the aforementioned dilemma is that sperm selection, an essential step in assisted reproductive technology, has remained at the morphological level while all other areas of medicine have advanced to the biochemical and even molecular levels. This enormous “gap” originates from the extremely strict requirements for a biochemical-level sperm-selection technology, that is it not only needs to detect the biochemical substances of sperm, but also cannot invade and harm sperm when detection, which is theoretically difficult and far beyond the state-of-the-art methods.

Overall, developing a non-invasive and damage-free technology to promote sperm selection from the primitive morphological level into the advanced biochemical level is extremely significant and long anticipated. The present invention addresses this need.

### SUMMARY OF THE INVENTION:

The present invention provides a Biochemical-Level Automatic-screening Smart droplet-TO-micro-hydrogels chip (BLASTO-chip) system microfluidic for automatic separation of live sperm and subsequent formation of single-sperm-encapsulated microhydrogels. The system includes a microfluidic chip and at least one pressure pump equipped with one or more syringes. The microfluidic chip includes at least one first channel for inflow of an aqueous phase, at least one second channel for inflow of a flowing oil phase, an outlet for the one or more single-sperm-encapsulated droplets to flow out, and a culture plate connected to the outlet for droplet collection. The aqueous phase includes sperm evenly dispersed in a solution. The at least one first channel and the at least one second channel are configured to create an interconnected cross-junction flow path to facilitate the mixing of the flowing oil phase and the aqueous phase, forming one or more single-sperm-encapsulated droplets. In the BLASTO-chip system, all the involved biochemical reactions occur outside the sperm cells, and the materials used are acknowledged to be noncytotoxic.

When a plurality of calcium sulfate nanoparticles is introduced into the culture plate and mixed with the one or more single-sperm-encapsulated droplets, droplet-to-hydrogel transformation is initiated and the single-sperm-encapsulated microhydrogels are formed. The microfluidic chip system selects biochemically active sperm with an accuracy of over 90%.

In an embodiment, the at least one pressure pump establishes connections between the at least one first channel and the at least one second channel to the one or more syringes.

In an embodiment, the flowing oil phase includes a flowing oil and a fluoro surfactant. It should be noted that any oil that does not blend with water can be used in the present invention. This may include other types of fluorinated oil, silicon oil, mineral oil, paraffin oil, or even other organic solvents, as long as they meet the standard of not mixing with water.

The solution in the aqueous phase includes an alginate salt such as alginate, sodium alginate, potassium alginate, or a combination thereof.

The fluoro surfactant includes perfluorooctanesulfonic acid (PFOS), perfluorooctanoic acid (PFOA), perfluorinated alkyl sulfonamido ethanols, fluorotelomer-based surfactants, perfluoropolyether-based surfactants.

In an embodiment, the sperm have a concentration ranging from 1×10⁵ cells/mL to 1×10⁶ cells/mL.

In an embodiment, the at least one first channel has a width ranging from 50-70 μm and a depth ranging from 80-100 μm, and the at least one second channel has a width ranging from 50-70 μm and a depth ranging from 80-100 μm.

In an embodiment, the at least one first channel has a flow rate in a range of 1-10 μL/min, the at least one second channel has a flow rate in a range of 1-10 μL/min.

In an embodiment, the one or more single-sperm-encapsulated droplets have a uniformed size ranging from 60-120 μm and a pH value in a range of 7.0 to 8.0.

Preferably, the one or more single-sperm-encapsulated droplets have a uniformed size of 100 μm.

In an embodiment, the sperm contained in the single-sperm-encapsulated microhydrogels are viable, and the single-sperm-encapsulated microhydrogels have a pH value in a range of 3.0 to 7.0.

In another aspect, the present invention also provides a method for improving the success rate of in vitro fertilization in a patient with asthenospermia, including processing semen samples and collecting sperm; preparing a continuous phase and a dispersed phase, with each added to a syringe of said microfluidic chip system; mixing the continuous phase and the dispersed phase to form one or more single-sperm-encapsulated droplets, the one or more single-sperm-encapsulated droplets are collected on a culture plate; adding calcium sulfate nanoparticles and cocultured them with the one or more single-sperm-encapsulated droplets for an incubation time to form single-sperm-encapsulated microhydrogels; adding an aqueous culture medium into the culture plate to make the microhydrogels diffuse into the aqueous culture medium, while the droplets remain unchanged; adding alginate lyase to dissolve the microhydrogels and release selected sperm; incubating the selected sperm in a fertilization medium for activation and subsequently injecting them into oocytes using microinjection. The method achieves a fertilization rate of at least 70% that is comparable to the fertilization rate obtained using sperm with normal quality.

In an embodiment, the aqueous phase comprises sperm evenly dispersed in a solution.

In an embodiment, the step of processing semen samples and collecting sperm further comprising subjecting the semen samples to density gradient centrifugation (DGC) or swim-up procedures to remove somatic cells and bacteria.

In an embodiment, the incubation time is in a range of 30 minutes to 3 hours.

In an embodiment, the selected sperm have a DNA fragmentation index of lower than 25%.

In an embodiment, the semen samples contain at least 1% live sperm with a progressive motility rate (PR) ranging from 0.1-100%. When the PR of the sperm is less than 10%, the number of the selected sperm reaches at least 1×10⁴.

In an embodiment, the method further including collecting oocytes from a subject, wherein the oocytes are in metaphase-II (MII) stage.

The BLASTO-chip system can select live sperm from samples containing only 1% live sperm, and the selected sperm are shown to be of good quality by evaluating their DNA integrity. To be more convincing, the present invention also demonstrates the reproductive nontoxicity of BLASTO-chip through sperm viability test and evaluations of the health of offspring from mice. The clinical relevance between sperm mobility and the number of selected sperm is also provided. Furthermore, BLASTO-chip technology is used to treat semen samples containing 10% live but 100% immobile sperm. The results reveals that the rates of fertilization, cleavage, early embryo and blastocyst formation are drastically increased. This clearly highlights the biosafety, therapeutic effect, and clinical application potential of the BLASTO-chip system.

### BRIEF DESCRIPTION OF THE DRAWINGS:

Embodiments of the invention are described in more details hereinafter with reference to the drawings, in which:

FIG. 1A depicts a schematic diagram of BLASTO-chip system;

FIG. 1B depicts diameter distribution of the generated droplets;

FIG. 1C depicts fluorescent images of the selected sperm after DAPI and PI staining;

FIG. 1D depicts the percentage of single sperm encapsulation at different sperm concentrations;

FIG. 1E depicts the distribution of hydrogels encapsulating different viable sperm and applicable range of hydrogels;

FIG. 2 depicts that when the droplet diameter is less than 60 μm, the tail of the sperm is curled or exposed outside the micro-hydrogels;

FIG. 3A depicts a sample subjected to density gradient centrifugation. FIG. 3B depicts an image of micro-hydrogels sinking to the bottom of a culture dish after adding the culture medium;

FIG. 4 depicts a schematic diagram of multiplexed BLASTO-chip by driving multiple chips with one pump;

FIG. 5A depicts the initial pH value and incubation time of the droplets affect

the formation of micro-hydrogels;

FIG. 5B depicts the quantity of formed hydrogels under different incubation time using samples containing 30% live sperm;

FIG. 5C depicts the quantity of formed hydrogels under different initial pH using samples containing 30% live sperm;

FIG. 5D depicts the regulation range of the selection efficiency of BLASTO-chip;

FIG. 6 depicts the experimental design for counting the number of micro-hydrogels;

FIG. 7A depicts microscopic images showing irregular droplets with dead sperm or no sperm. FIG. 7B depicts the quantity of formed hydrogels under different initial pH using samples containing 100% dead sperm. FIG. 7C depicts the quantity of formed hydrogels under different incubation time using samples containing 100% dead sperm;

FIG. 8 depicts motility and viability of sperm when exposed to alginate oligosaccharides (AOs), calcium ions, and alginate lyase;

FIG. 9A depicts a workflow of the mouse ICSI-embryo transfer experiment;

FIG. 9B depicts 2-cell embryos, where each sperm in each embryo was selected by using the BLASTO-chip system;

FIG. 9C depicts the body weight change of the first-generation mice populations;

FIG. 9D depicts the average body weights of six-week F2 mice. Each group represents mice born by the same parent female mouse;

FIG. 10 depicts the reproductive performance of the BLASTO-chip assisted F1 mice, including each female mouse's baby/litter, fetal mortality, female/male proportion and viability;

FIG. 11 depicts the pH of bulk solutions containing sperm with different motility at different timepoint;

FIG. 12 depicts the detailed theoretical mechanism for activation of sperm motility;

FIG. 13A depicts the number of formed hydrogels/selected sperm when using BLASTO-chip to treat semen samples with PR of 5%, 25% and 50%. One-way ANOVA. **P≤0.01, ***P≤0.001, ****P≤0.0001;

FIG. 13B depicts the heat map displayed the quantity of hydrogels produced using sperm samples with various PR percentages (5%, 25%, and 50%) during various incubation times (t=0.5 h, 1.5 h, and 2.5 h);

FIG. 13C depicts selection of highly active sperm by reducing the incubation time;

FIG. 14 depicts the preparation of a series of pooled samples (I to VII) in which the percentage of viable sperm ranged from 1% to 100%;

FIG. 15A depicts the results of eosanidine black staining of samples from groups I to VII in FIG. 14;

FIG. 15B depicts the percentage of viable sperm before/after selection;

FIG. 16A depicts sperm DNA fragmentation index (DFI) of chosen sperm. The DFI values were determined by SCD;

FIG. 16B depicts the DFI values of three semen samples before and after the selection via BLASTO-chip. The DFI values were determined by SCD;

FIG. 17A depicts procedure for injection of selected sperm into human oocytes by ICSI;

FIG. 17B depicts the results of eosanidine black staining of samples before and after selection;

FIG. 18 depicts the alignment of the BLASTO-chip selection process with the clinical timeline of ICSI/IVF;

FIG. 19 depicts baseline data of the oocytes donors, including antral follicle count, age, infertility year, BMI, concentrations of LH, FSH, E2 and AMH;

FIG. 20A depicts fertilization rate, cleavage rate, early embryo rate, and blastocyst rate of the samples in which sperm were screened using the present BLASTO Chip system; and

FIG. 20B depicts the individual oocytes' developmental outcomes following ICSI.

### DETAILED DESCRIPTION:

In the following description, Biochemical-Level, Automatic-screening, Smart droplet-TO-hydrogel chip (BLASTO-chip) system and method for sperm selection are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Over the decades, sperm selection has primarily depended on the primitive physical conditions (morphology or motility) of sperm, despite advancements in other branches of clinical medicine that have delved into biochemical or even molecular levels. This substantial “gap” presents formidable challenges for infertile patients, particularly those grappling with severe and total asthenozoospermia.

In order to fill the huge gap, the present invention develops a system called Biochemical-Level, Automatic-screening, Smart droplet-TO-hydrogel chip (BLASTO-chip) for sperm selection. The system includes a microfluidic chip and at least one pressure pump equipped with one or more syringes. The microfluidic chip includes at least one first channel for inflow of an aqueous phase, at least one second channel for inflow of a flowing oil phase, an outlet for the one or more single-sperm-encapsulated droplets to flow out, and a culture plate connected to the outlet for droplet collection. The aqueous phase includes sperm evenly dispersed in a solution. The at least one first channel and the at least one second channel are configured to create an interconnected cross-junction flow path to facilitate the mixing of the flowing oil phase and the aqueous phase, forming one or more single-sperm-encapsulated droplets. In the BLASTO-chip system, all the involved biochemical reactions occur outside the sperm cells, and the materials used are acknowledged to be noncytotoxic.

When a plurality of calcium sulfate nanoparticles is introduced into the culture plate and mixed with the one or more single-sperm-encapsulated droplets, droplet-to-hydrogel transformation is initiated and the single-sperm-encapsulated microhydrogels are formed. The microfluidic chip system selects biochemically active sperm with an accuracy of over 90%. The BLASTO-chip system can sense the pH change caused by sperm's breathing products and select biochemically active sperm with an accuracy of over 90%, and its selection efficiency could be flexibly tuned by nearly 10 folds.

The flowing oil phase may contain a flowing oil and a fluoro surfactant. The flowing oil may be fluorinated oil, silicon oil, mineral oil, paraffin oil, or even other organic solvents, as long as they meet the standard of not mixing with water. For instance, the fluorinated oil may be perfluorohydrocarbons Is (PFHCs), perfluoropolyethers (PFPEs), or perfluorinated alkyl esters (PFAEs). The fluoro surfactant may be perfluorooctanesulfonic acid (PFOS), perfluorooctanoic acid (PFOA), perfluorinated alkyl sulfonamido ethanols, fluorotelomer-based surfactants, perfluoropolyether-based surfactants, etc. The solution in the aqueous phase may contain an alginate salt such as alginate, sodium alginate, potassium alginate, or a combination thereof.

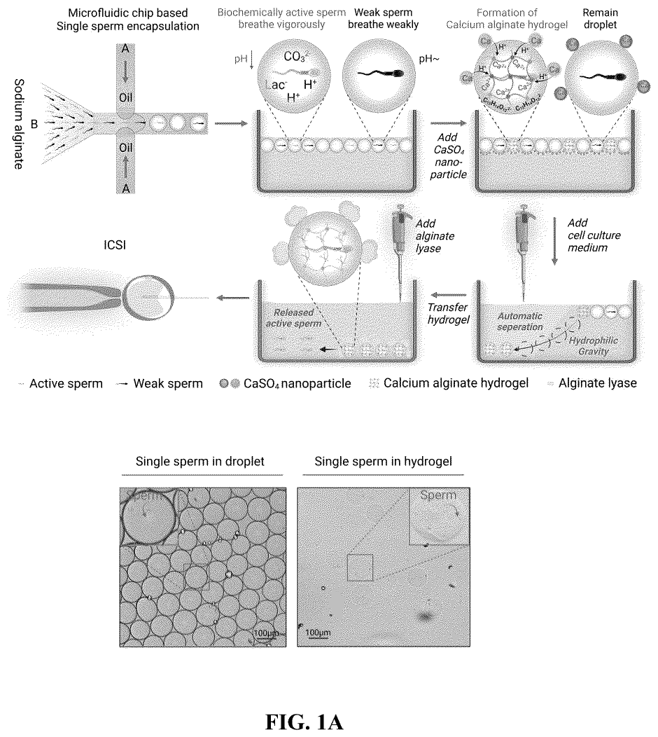

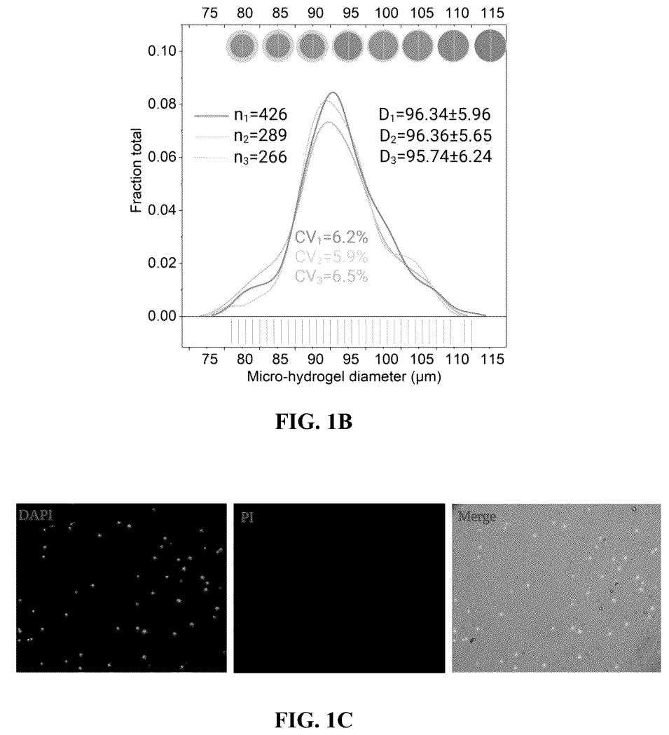

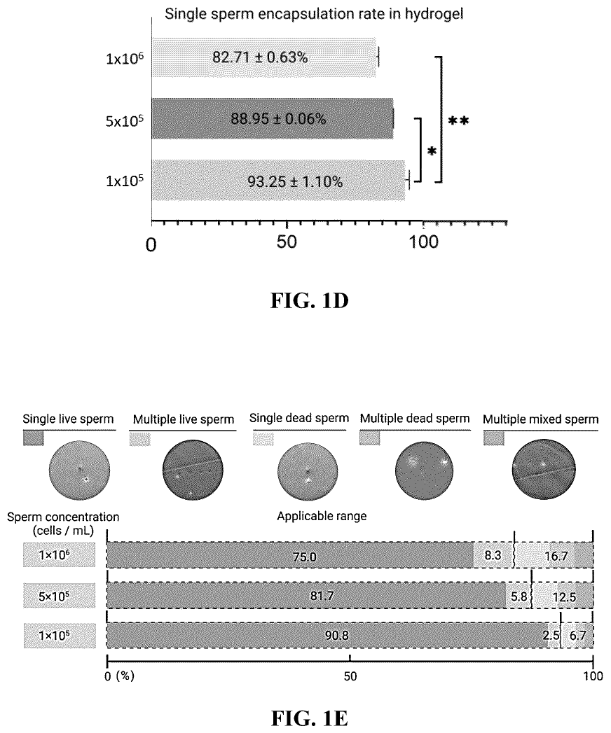

The first step of the BLASTO-chip system entails encapsulating individual sperm into droplets, and ensuring that each droplet contains either one sperm or none at all. Turning to FIG. 1A, the present invention uses a “cross-junction” microfluidic chip to generate water-in-oil droplets and encapsulate a single-sperm in this microreactor by connecting to a microinjection pump. The chip consists of two inlets (channel A), one outlet (channel B), and an observation chamber for droplet collection. Toward channel A, the flow phase consisting of fluorinated oil (biosafe and widely applied in cell culture) mixed with 1% fluoro surfactant is injected. Toward channel B, the aqueous phase consisting of sperm evenly dispersed in sodium alginate solutions (biosafe and widely adopted in three-dimensional (3D) culture of oocytes and embryos) is injected. By subtly optimizing the flow rate of these channels, a large amount of microdroplets can be prepared in a relatively short time, with each droplet contains either one or no sperm, i.e. single-sperm droplet. The flowing oil phase used in the present invention is fluorinated oil, proven to be biosafe and widely applied in cell culture. As for the aqueous phase, alginate is also biosafe and widely adopted in the three-dimensional (3D) culture of oocytes and embryos.

In one embodiment, the sperm selection system is based on a microfluidic chip with a cross-junction flow path, where the width and depth of the channels are 60 μm and 90 μm, respectively. By regulating the flow rate of the flowing oil phase in the channel A and the flow rate of the aqueous phase in the channel B, it is possible to consistently and rapidly generate a large number of droplets with uniformed size.

Turning to FIG. 1B, the generated droplets were evenly dispersed and their sizes are uniformed with a CV of 5.9% to 6.5%. The diameter of the generated droplets in the present invention is approximately 60-120 μm.

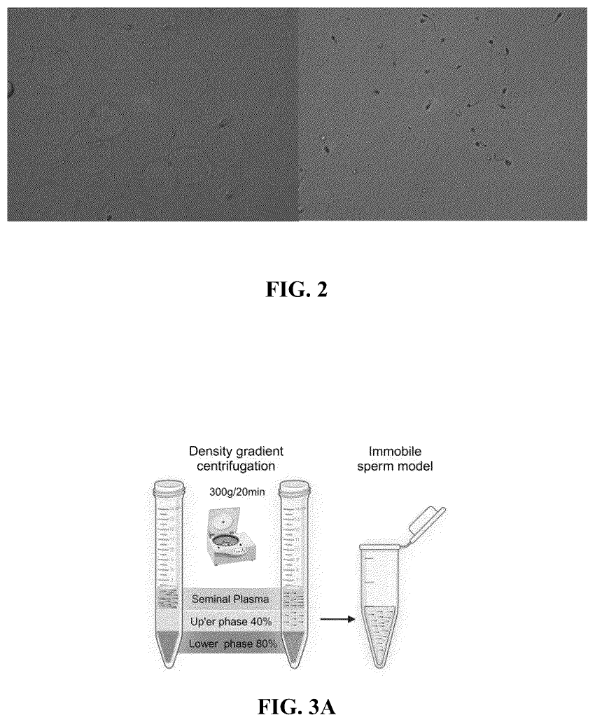

It is important to generate microdroplets with appropriate diameters. For small droplets with a diameter of less than 60 μm, sperm' tails would be curled or exposed outside the micro-hydrogel (FIG. 2). For large droplets with a diameter over 150 μm, sperm must undergo extensive metabolic processes so that the droplets can transform into micro-hydrogels, which decreases the capture efficiency.

Preferably, the diameter of the generated droplets in the present invention is approximately 85-100 μm.

More preferably, the diameter of the generated droplets in the present invention is approximately 90 μm.

In one embodiment, the concentration of sperm is in a range of 1×10⁵ cells/mL to 5x105 cells/mL. Based on the flow rates of the channels and the concentration of sperm (e.g., 1×10⁵ cells/mL), it can be estimated that the number of the droplets is approximately 10 times that of sperm. This means that, statistically, 90% of the droplets contain no sperm while the other 10% contain one sperm.

The biochemical activity and metabolic products of sperm can achieve the effect of transforming liquid droplets into hydrogels. In one embodiment, the samples with 100% live sperm by swim-up method and samples with 100% dead sperm by at 85° C. for one hour were obtained. Both live and dead sperm are stained with DAPI (4′,6-diamidino-2-phenylindole) and PI (propidium iodide). DAPI is used to visualize the location and number of nuclei in a sample, while PI is used to stain cells that have lost membrane integrity, such as dead or apoptotic cells. After washing away excess dyes, live and dead sperm are mixed to obtain samples with 50% live sperm. The BLASTO-chip is used to disperse the stained sperm into microdroplets. The microdroplets are incubated at 37° C. with 5% CO₂ for 120 minutes, and then mixed with CaSO₄ nanoparticles to induce the formation of hydrogels. The hydrogels are then separated automatically. Finally, the fluorescence of the stained sperm captured by the present BLASTO-chip system is observed. As shown in FIG. 1C, the single-sperm encapsulated in the hydrogels are all DAPI-stained sperm, proving that only live sperm can initiate the droplet-to-hydrogel transformation.

The singularity of sperm in the droplets is vital for successful sperm selection. If there are multiple sperm within a droplet, it will form hydrogel as long as live sperm exists, regardless of the viability of other sperm. Consequently, the selection efficiency for live sperm will be affected. Therefore, it is crucial to maintain a high ratio of single-sperm encapsulation.

Referring to FIG. 1D, sperm samples with different concentrations (1×10⁵, 5×10⁵, and 1×10⁶ cells/mL) are prepared and the proportions of the generated hydrogels containing single live sperm, multiple live sperm, single dead sperm, multiple dead sperm, multiple mixed sperm are measured. The hydrogels containing single live sperm are ideal and those containing multiple live sperm are applicable. As shown in FIG. 1E, when using sperm concentration of 1×10^{5,} the single-live-sperm proportion and the applicable proportion are highest (90.8% and 93.3%). These experimental values are statistically higher than the theoretical values (approximately 5%) based on the Poisson distribution. In comprehensive consideration of the percentage of single-sperm and the consumed time for obtaining enough hydrogels, 1×10⁵ cells/mL is chosen for subsequent experiments.

All the reactants and substances used in the water-in-oil droplet system are non-toxic to sperm, and the designed biochemical reactions all occur outside the sperm. The single-sperm droplets are incubated for 1.5 to 2.5 hours at 37° C. and 5% CO₂. During the incubation process, live and biochemically active sperm will breathe and secret many respiration products, which are mainly lactic acid due to the anaerobic respiration of human sperm. Whereas, dead or inactive sperm will produce little lactic acid. Consequently, the pH value inside the droplets that contain live and active sperm will be considerably lower than those containing dead or inactive sperm. After incubation, calcium sulfate (CaSO₄) nanoparticles are added to the system and diffused into the droplets. The droplets with a lower pH value then resolve CaSO₄ nanoparticles and release soluble calcium ions (Ca²⁺) into the droplets. The presence of certain amount of soluble Ca²⁺ can induce polymer crosslinking of alginate, and the droplets with lower pH will eventually form alginate hydrogels.

After that, an aqueous culture medium is added into the plate, and due to the gravity and hydrophobic effect, the hydrogels will automatically diffuse into the aqueous culture medium, whereas the droplets remain unchanged. By this way, the present invention can achieve high-throughput capture and automatic separation of live and active sperm at the biochemical level. For subsequent fertilization, alginate lyase can be used to resolve the hydrogel and release selected sperm. The selected sperm are incubated in the fertilization medium for activation and then injected into the oocytes via ICSI.

In another aspect, the present invention provides a method for improving the success rate of in vitro fertilization in a patient with asthenospermia, including processing semen samples and collecting sperm; preparing a continuous phase and a dispersed phase, with each added to a syringe of said microfluidic chip system; mixing the continuous phase and the dispersed phase to form one or more single-sperm-encapsulated droplets, the one or more single-sperm-encapsulated droplets are collected on a culture plate; adding calcium sulfate nanoparticles and cocultured them with the one or more single-sperm-encapsulated droplets for an incubation time to form single-sperm-encapsulated microhydrogels; adding an aqueous culture medium into the culture plate to make the microhydrogels diffuse into the aqueous culture medium, while the droplets remain unchanged; adding alginate lyase to dissolve the microhydrogels and release selected sperm; incubating the selected sperm in a fertilization medium for activation and subsequently injecting them into oocytes using microinjection. The method achieves a fertilization rate of at least 70% that is comparable to the fertilization rate obtained using sperm with normal quality.

In an embodiment, the aqueous phase comprises sperm evenly dispersed in a solution.

In an embodiment, the step of processing semen samples and collecting sperm further comprising subjecting the semen samples to density gradient centrifugation (DGC) or swim-up procedures to remove somatic cells and bacteria.

In an embodiment, the incubation time is in a range of 30 minutes to 3 hours.

In an embodiment, the semen samples contain at least 1% live sperm with a progressive motility rate (PR) ranging from 0.1-100%. When the PR of the sperm is less than 10%, the number of the selected sperm reaches at least 1×10⁴.

Preferably, the semen samples contain less than 10% live sperm with a progressive motility rate (PR) of less than 70%.

Comprehensively, the sperm selection process in the BLASTO-chip system is purely biochemical and completely independent of the motility of the sperm. All the substances used in the system are proven to be non-toxic, and the designed biochemical reactions all occur outside the sperm cells. Therefore, the proposed strategy is theoretically non-invasive and biosafe.

### EXAMPLE

### EXAMPLE 1-1

### Source of materials

Mouse sperm and mouse oocytes were obtained from Laboratory Animal Center, Huazhong University of Science and Technology, China. The oocytes used in the present invention were all in vitro matured (IVM) and the sperm used in the model were of very low quality. Human Normal Prostate Epithelial Cells-RWPE-1 cells (KMCC-001-0066) were purchased from Shanghai Kunmeng Biotechnology Co., LTD, China.

The sources of the chemicals, peptides, and recombinant proteins used in this invention were listed in the Table 1 below. Notably, all the reactants of the water-in-oil droplet system is non-toxic to sperm.

TABLE 1

| REAGENT or RESOURCE | SOURCE | IDENTIFIER |
|---|---|---|
| PDMS | Sigma-Aldrich | Cat#57309 |
| SU8-2050 | MicroChem | N/A |
| HFE-7500 | 3M Novec | N/A |
| PBS buffer | Sigma-Aldrich | Cat# E607008 |
| MEM | Sigma-Aldrich | Cat#M4655 |
| CaCl2 | Sigma-Aldrich | Cat#931144 |
| Glucose | Sigma-Aldrich | Cat#D9434 |
| L-Glutamine | Sigma-Aldrich | Cat#G5792 |
| CaSO₄ nanoparticle | This paper | N/A |
| Fluorosurfactant | RANBIOTECH | Cat#008 |
| Sodium alginate | Sigma-Aldrich | Lot#MKCF5032 |
| H₂O₂ | Sigma-Aldrich | Cat#655104 |
| RPMI-1640 medium | Sigma-Aldrich | Cat#R8758 |
| 1H,1H,2H,2H-perfluorooctanol | Sigma-Aldrich | Cat#924539 |
| Alginate lyase | Sigma-Aldrich | Cat#A1603 |
| PMSG | NSHF | N/A |
| HCG | NSHF | N/A |
| Hyaluronidase | Sigma-Aldrich | Cat#H1115000 |
| G1-Plus | | |
| HTF | Sigma-Aldrich | Cat#MR070 |
| hCG | Livzon | N/A |
| | Pharmaceutical | |
| | Group | |
| Blastocyst culture medium | COOK | N/A |
| Embryo culture medium | COOK | N/A |
| G1-PLUS | Vitrolife | Cat#10136 |
| Fertilization medium | COOK | N/A |
| DAPI | Sigma-Aldrich | Cat#D9542 |
| PI | Beyotime | Cat#ST511 |
| Eosin-aniline | BRED life science | BRED-014 |
| SCD assay | BRED life science | BRED-002 |

### EXAMPLE 1-2

### Sperm Staining

Live sperm samples were stained for 15 minutes with DAPI (4′,6-diamidino-2-phenylindole) (Sollerbauer, Beijing, China), and dead sperm samples were stained for 10 minutes with PI (Beyotime, Shanghai, China) (1000:1), followed by three 5-minute washes with PBS. Fluorescent images were captured with a fluorescence microscope (SUNNY ICX41). The viability of sperm was assessed using eosin-aniline (BRED life science, Shenzhen, China), which can enter damaged cell membranes for staining but not live cell membranes, which resist dye penetration. Damaged cell membranes allow non-transmissive dyes to enter the membrane for staining, whereas live cell membranes can resist dye entry (WHO). The sperm solution and dye were thoroughly mixed at a volume ratio of 1:1 and stained for 30 seconds. Spermatozoa with white heads were considered alive, whereas those with dark pink or red heads were considered non-viable. (Viability of spermatozoa =number of living sperm/number of all detected sperm). The sperm chromatin dispersion (SCD) test was used to measure sperm DNA fragmentation²³⁻²⁴. Sperm with intact DNA produced a distinct halo after deformation and removal of nucleoproteins, but sperm containing DNA fragments did not. The existence or absence, as well as the size of the halo, can be used to measure the degree of DNA integrity, according to this principle. For further information, refer to the kit instructions (BRED life science, Shenzhen, China).

### EXAMPLE 1-3

### Mouse Experiments

All mouse experiments were performed in accordance with the guidelines of the Institutional Animal Care and Use Committee (IACUC) of Huazhong University of Science and Technology, Wuhan, China. Healthy KM female mice at 7-8 weeks were induced superovulation with 7.5 IU pregnant mare serum gonadotropin (PMSG) (NSHF, Ningbo, China), followed by 7.5 IU human chorionic gonadotropin (HCG) (NSHF, Ningbo, China) after 44-48 h. Cumulus oocyte complexes (COCs) were retrieved from their oviducts at 14 h of hCG administration. Oocytes were employed in following experiment after being treated with hyaluronidase. Sperm were obtained from the epididymal tail of male KM mice aged 8-9 weeks, and then immediately capacitated in HTF medium for 15 min. Using a microfluidic system, high-quality mouse sperm were selected, and their tails were severed for five seconds though ultrasonic treatment. The ultrasonic treatment did not cause damage to the sperm structure, nor does it affect the potential for subsequent fertilization capacity. These selected mouse sperm were subjected to PIEZO-ICSI with mouse oocytes under a Nikon inverted microscope. The sperm heads were placed in a 5% PVP droplet, and the oocytes were situated in the microinjection droplet (CZB.H). A microinjection needle aspirated one or multiple sperm heads. Based on experience, approximately 10 sperm heads can be accumulated in a pipette with an interval of about 5 μm, negating the need to return to the sperm-PVP droplet between each injection. A slight negative pressure was introduced inside the microinjection needle and a piezoelectric pulse (intensity=5, frequency=1) was applied using the Eppendorf PiezoXpert (Germany). Concurrently, the microinjection needle was gently advanced, piercing through the zona pellucida swiftly to enter the perivitelline space, halting any further pressure. Then, the needle's tip was steadily advanced to the opposite side of the oocyte, ensuring the pipette remained anchored. Progression halted when the tip advanced to about 95% of the oocyte's diameter, which takes 2-5 seconds. Subsequently, the piezo preset channel (foot switch) was adjusted to a gentler setting (for example, intensity=1, frequency=1). With zero net pressure (or very minimal negative pressure) maintained inside the microinjection pipette, a single piezoelectric pulse was administered. The sperm head was deposited into the cytoplasm while applying a slight positive pressure in the microinjection pipette. The introduction of extraneous medium was minimized, ensuring cytoplasm was not aspirated into the pipette. Once the sperm was deposited, the needle was smoothly retracted from the oocyte. The membrane should revert to its initial position and seal. If the vitelline membrane sticks to the pipette, pause briefly during withdrawal. Post-injection, oocytes were rinsed 4-6 times in the culture medium, transferred into G1 Plus medium, and then cultured in an incubator with a temperature of 37° C. and 5% CO₂ atmosphere.

A pseudopregnant mice, female mice aged 8 weeks was mated with male mice having vas deferens removed. The mouse's fallopian tubes were then found by routine surgery following anesthesia. A total of 21 zygotes in 2-cell stage were transplanted into the fallopian tube incision using a microtubule at a time. After the wound was stitched up, the mice were moved to a suitable place where they could be cultured until their pups were born. The mice pups were weighed weekly from their first week of life. At the sixth week, male and female pups were raised in separate cages. After 8 weeks, a male and female mouse were caged together to produce the next generation of mice.

### EXAMPLE 1-4

### Human Oocytes Collection, IVM and ICSI

Human semen samples were from the Reproductive Medicine Center of Changsha Women's and Children's Hospital, China. The informed consent was obtained from all participants after the nature and possible consequences of the studies were explained.

A total of 64 immature human oocytes in germinal vesicle (GV) or metaphase-I (MI) phase were collected from 23-35 years old patients who underwent IVF/ICSI treatments. Controlled ovarian stimulation was performed, and transvaginal ultrasonography was used to guide oocyte collection 36 hours following hCG (Livzon Pharmaceutical Group, Zhuhai, China) injection. After removing oocyte granulosa cells using hyaluronidase, oocytes in metaphase-II (MII) stage were inseminated by ICSI and those at MI and GV stages were donated for research. The immature oocytes were cultured in IVM medium for 24 hours. Then MII oocytes with the first polar body extrusion were picked up for the further study.

To ensure consistent oocyte quality, each oocyte was evaluated morphologically and oocytes of poor quality were discarded. A total of 44 available oocytes were randomly divided into microfluidic group and control group, and then ICSI was performed under Nikon inverted microscope with sperm from M-group and C-group, respectively. Finally, these oocytes were cultured in media (used during the culture of embryos at the gamete, fertilization, cleavage, and blastocyst stages) provided by COOK (Bloomington, IN, USA) at 37° C., 6% CO₂ and 5% O₂. The fertilization rate (the proportion of zygotes to the number of oocytes at MII stage) was calculated for 16-18 hours after ICSI. The cleavage rate (the proportion of early embryos to the number of oocytes at MII stage) was calculated after 72 hours. The blastocyst rate (the proportion of blastocyst number to the number of oocytes at MII stage) was assessed 5-6 days after ICSI.

### EXAMPLE 2

### Fabrication of Microfluidic Devices and Chip

The polydimethylsiloxane (PDMS) (Dow Corning, MI) microfluidic device was fabricated as well-established soft photolithography procedures. The mask design was created with AutoCAD software and then printed in high quality (25 400 dpi). Following the manufacturer's instructions, SU8-2050 (MicroChem, Westborough, MA) was spin-coated on a silicon wafer to make a master mold at a depth of 90 μm. The precursor for PDMS was mixed with a crosslinker in a 10:1 ratio, poured to a prepared master mold, and then incubated for 120 minutes at 65° C. The PDMS duplicate was then removed from the mold, pierced with a 1 mm blunt hypodermic needle, and plasma-bonded to another PDMS slab. Next, the device was salinized using 1% (Tridecafluoro-1,1,2,2-Tetrahydrooctyl)-1-Trichlorosilane in HFE-7500 (3M Novec Engineering Fluid HFE-7500), fluorinated oil. The oil was put into microfluidic channels at a level sufficient to completely wet the whole microfluidic network. The device was then maintained at 95° C. for at least 30 minutes. To restore microchannel hydrophobicity, these chips could be kept in an oven at 65° C. for a couple of days. The device included two inlets for the disperse continuous phase (with sample) and continuous phase (with oil) separately, one outlet for droplet collection. Polyethylene tubing was used to connect channels to glass syringes (Gaoge, shanghai, China), which were mounted on the microinjection pumps (CHEMYX, F100X) (FIG. 1A). The dispersed phase liquid was put into a 250 μl syringe, and then the syringe was connected to one microinjection pump via the polyethylene tubing. The continuous phase liquid was put into a 1000 μl syringe, and then the syringe was connected to another microinjection pump via the polyethylene tubing.

### EXAMPLE 3

### Collection and Processing of Human Sperm Samples

Human sperm samples were discarded sperm collected after the regular therapies. Fresh semen samples were processed by a direct swim-up technique using standard methods (WHO). By aspirating the uppermost media, sperm samples with 100% motility and viability were obtained. On the other hand, the dead sperm samples were prepared by re-suspending the remaining semen in phosphate-buffered saline (PBS) (Sangon Biotech, shanghai, China) three times, centrifuged at 300 g for 10 minutes. The supernatant was discarded and the pellets were heat-treated at 85 degrees for 1 hour.

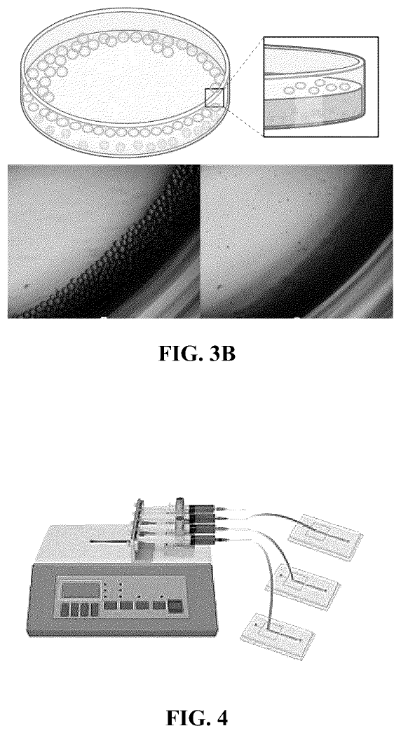

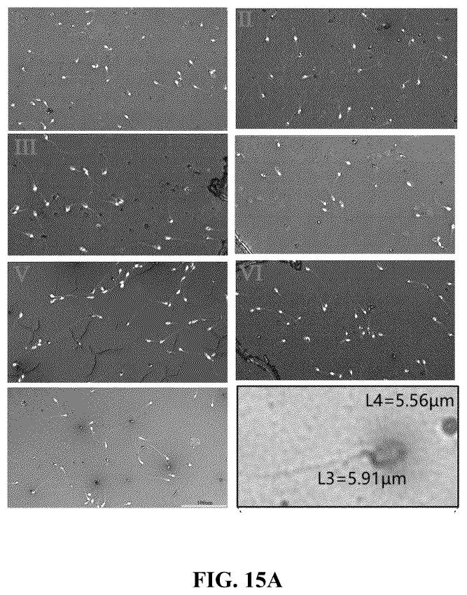

Sperm samples with 100% motility and vitality were blended with varying amounts of dead sperm samples to create sperm samples with 70%, 50%, 30%, 20%, 10%, and 1% vitality (FIG. 14). The model of entirely immobile sperm was based on the concept of density gradient centrifugation (FIG. 3A), suggesting that there exists a distinction in the motility and trajectory between normal sperm and immobile sperm within a gradient solution column.

Referring to FIG. 3A, the fresh sperm samples underwent processing using standard differential density gradient centrifugation procedures. The sperm was carefully drawn up into the midst of the gradient solution, taking care to avoid disturbing the bottom sediment. Upon observation under a microscope, it became apparent that the sperm were largely immotile, with only a portion remaining viable following eosin-nigrosin staining. After combining a particular amount of dead sperm samples, a 10% model of live yet immotile sperm was created. The sperm concentrations were calculated by taking the average of three counts from a hemocytometer plate.

The presence of somatic cells and bacteria in semen samples is not rare. Under normal circumstances, the ratio of somatic cells (primarily white cells and epithelial cells) to sperm in semen samples ranges from 0 to 1%, while the ratio of bacteria to sperm ranges from 0 to 0.001%. In the present invention, both bacteria and somatic cells immersed in sperm can be selected out by the BLASTO-chip due to their energy metabolism process, as shown in Table 2.

TABLE 2
Influence of bacteria and somatic cells
on sperm selection by the BLASTO-chip

| | | | Number of | Number of |
|---|---|---|---|---|
| | | Number | bacteria per | somatic cells |
| | Sample | of | HPF after | per HPF after |
| Groups | composition | hydrogels | selection^{a} | selection |
| Control | Dead sperm | 0 | 0 | 0 |
| Group 1 | Dead sperm + | 442 | 106 | 0 |
| | bacteria | | | |
| Group 2 | Dead sperm + | 430 | 0 | 12 |
| | somatic cells | | | |
| Group 3 | Dead sperm + | 490 | 112 | 15 |
| | bacteria + | | | |
| | somatic cells | | | |

^{a}HPF is the abbreviation for high power field

Toward such interference, the issue can be addressed in a categorized manner:
i) For the majority of normal semen samples, the proportion of somatic cells relative to the sperm count is very low (0-1%). Even if they can form hydrogels along with the sperm, their proportion remains low (0-1%), which is even much lesser than the hydrogels formed mistakenly by dead sperm (about 5%). Hence, these somatic cells are believed to only cause the selection correctness of the BLASTO-chip to decrease by less than 1%, which is very slight and will not have any considerable effect on the subsequent treatment. As for the bacteria, their number relative to the sperm is extremely low (less than 0.001%), and can thus be neglected.
ii) For some patients with severe infections or inflammations, their semen might contain a significantly increased number of white cells and bacteria. This may result in a higher number of white cells and bacteria in the sperm after BLASTO-chip selection, further affecting the subsequent treatment. In such cases, users are recommended to initially subject the semen sample to density gradient centrifugation (DGC) or swim-up procedures to remove somatic cells and bacteria, and subsequently use the BLASTO-chip to select biochemically active sperm.

TABLE 3
Eliminating the influence of bacteria and somatic cells on sperm selection
by treating semen samples with Swim up/DGC prior to BLASTO-chip

| | | | Number of | Number of | |
|---|---|---|---|---|---|
| | | Sample | bacteria | somatic | |
| | | treatment | per HPF | cells per | Number |
| | Sample | Before | after | HPF after | of |
| Groups | composition | BLASTO-chip | BLASTO-chip | BLASTO-chip | hydrogels |
| Group 1 | Dead sperm + | Swim up | 0 | 0 | 0 |
| | bacteria | | | | |
| Group 2 | Dead sperm + | Swim up | 0 | 0 | 0 |
| | somatic cells | | | | |
| Group 3 | Dead sperm + | Swim up | 0 | 0 | 0 |
| | bacteria + | | | | |
| | somatic cells | | | | |
| Group 4 | Dead sperm + | DGC | 0 | 0 | 0 |
| | bacteria | | | | |
| Group 5 | Dead sperm + | DGC | 0 | 0 | 0 |
| | somatic cells | | | | |
| Group 6 | Dead sperm + | DGC | 0 | 0 | 0 |
| | bacteria + | | | | |
| | somatic cells | | | | |

^{a} HPF is the abbreviation for high power field

Experimental results in Table 3 demonstrated that by DGC or swim up, the somatic cells and bacterial were completely eliminated. In addition, the strategy (DGC/Swim up+BLASTO-chip) were applied to a clinical semen sample exhibiting severe infection (1×10⁸ CFU/mL) and leukocytospermia (5×10⁶ cells/mL). Experimental results in Table 3 showed that only sperm were finally selected out, demonstrating the clinical practicability of the strategy toward this specified group of patients.

### EXAMPLE 4

### Minimal Threshold of Sperm Quality

The exploration of the minimal threshold of sperm quality for selecting a sufficient number of sperm using the BLASTO-chip in both IVF and ICSI applications is ongoing. Typically, when the progressive motility (PR, %) ranges from 10 to less than 32, IVF (in vitro fertilization) was generally employed. When PR was between 1 and less than 10, either IVF or ICSI (intracytoplasmic sperm injection) could be utilized, whereas ICSI was recommended when PR was less than 1. For IVF, the required sperm count per oocyte was approximately 5,000, translating to a total sperm count ranging from 20,000 to 75,000 (for 4-15 oocytes).

To obtain a sufficient number of sperm, making adjustments to the parameters of the BLASTO-chip was essential for obtaining a sufficient number of sperm: (1) The channel flow rate of the BLASTO-chip was increased by 10-fold (10 μL/min) and the sperm sample concentration was raised to 1×10⁶ cells/mL, allowing for the generation of more droplets; and (2) As depicted in FIG. 4, a single pressure pump with its associated tubing could simultaneously drive multiple chips, enabling paralleled and multiplexed selection. Given that such pressure pump (Suzhou Xunfei Scientific Instrument Co., LTD, XFP04-B) had four channels, three chips were concurrently driven to enhance the selection efficiency. The BLASTO-chip system was then used to select sperm from semen samples of varying quality.

Experimental results in Table 4 indicated that for IVF, the BLASTO-chip required semen samples to have had a PR of over 5% (increasing the number of chips and applying prior density gradient centrifugation could further reduce the sperm motility requirement, as shown in Table 5). On the other hand, for ICSI, the BLASTO-chip imposed no constraints on sperm motility; as long as there were live and biochemically active sperm, they could be selected by the BLASTO-chip for subsequent ICSI.

TABLE 4
The number of selected sperm by BLASTO-chip
toward semen samples with different quality

| | | PR of | Estimated |
|---|---|---|---|
| | | the semen | number of |
| | ART | sample | selected |
| Groups (PR, %) | treatment | (PR, %) | sperm |
| Normal motility semen (>32) | IUI/IVF | 50 | ~6 × 10⁴ |
| Mild asthenospermia (20~32) | IUI/IVF | 30 | ~6 × 10⁴ |
| Moderate asthenospermia (10~20) | IUI/IVF | 10 | ~4 × 10⁴ |
| Severe asthenospermia (1~10) | IVF/ICSI | 5 | ~3.5 × 10⁴ |
| Extremely severe | ICSI | <1 | ~1 × 10⁴ |
| asthenospermia (<1) | | | |

^{a} The number of selected sperm was estimated by counting the number of hydrogels per field of view.

TABLE 5
The impact of density gradient centrifugation prior to BLASTO-
chip selection on the number of sperm selected by the BLASTO-chip

| | | PR of | Estimated |
|---|---|---|---|
| | | the semen | number of |
| | ART | sample | selected |
| Groups (PR, %) | treatment | (PR, %) | sperm |
| Normal motility semen (>32) | IUI | 50 | ~8 × 10⁴ |
| Mild asthenospermia (20~32) | IUI/IVF | 30 | ~8 × 10⁴ |
| Moderate asthenospermia (10~20) | IUI/IVF | 10 | ~6 × 10⁴ |
| Severe asthenospermia (1~10) | IVF/ICSI | 5 | ~6 × 10⁴ |
| Extremely severe | ICSI | <1 | ~4 × 10⁴ |
| asthenospermia (<1) | | | |

^{a} The number of selected sperm was estimated by counting the number of hydrogels per field of view.

### EXAMPLE 5

### Preparation of Continuous and Dispersed Phases

The continuous phase consisted of a fluorinated oil blend with a 1% fluorosurfactant admixture. The dispersed phase was prepared without an acid-base buffer. The following ingredients were used: 1×MEM, 4 mM L-Glutamine, 5 mM CaCl₂, and 4.5 g/L glucose. The pH value of the dispersed phase was measured using a pH meter, and sodium hydroxide was added as necessary to achieve various initial pH values (pH=7.2, pH=7.5, and pH=7.8). Additionally, the sperm suspension was dispersed in this medium, and subsequently mixed with 1% (w/v) sodium alginate.

### EXAMPLE 6

### Fabrication of Single-Sperm-Encapsulated Droplets and Micro-Hydrogels

The chip from Example 2 acted as a microreactor, generating water-in-oil droplets (approximately 100), each encapsulating a single-sperm. The detailed steps are as follows:

The sperm suspension from Example 3 was effectively dispersed in the dispersed phase and subsequently mixed with 1% (w/v) sodium alginate. To prevent the formation of localized calcium alginate that could potentially obstruct the channels of the chip, it is crucial to carefully ensure thorough mixing of the dispersed phase liquid.

Following that, two phases were added to different syringes, which were then interconnected to the PDMS chip and two microinjection pumps as mentioned in Example 2. Monodispersed droplets (aqueous phase in oil) with an average diameter of about 100 μm could be generated at a rate of 8 μl/min: 1 μl/min for the continuous phase and the dispersed phase, respectively, by regulating the microinjection pump (FIG. 1B). The droplets were collected into culture plates via polyethylene tubing, and then cultured in an incubator with 37° C. and 5% CO₂ atmosphere. Meanwhile, CaSO₄ nanoparticles were suspended in fluorinated oil, ultrasonically dispersed at 70% amplitude for 30 min, and then purified by centrifugation or filtration. After a period of droplets incubation (1.5 h, 2.0 h and 2.5 h), CaSO₄ nanoparticles were added to the culture plate and cocultured with droplets for 30 minutes. Due to the accumulation of sperm acid metabolites, free calcium ions (Ca²⁺) can dissociate from calcium sulfate nanoparticles at a low pH value. After that, Ca²⁺ cross-linked with sodium alginate, and the calcium alginate micro-hydrogels were generated accordingly.

The aforementioned emulsions were transferred to a 1.5 ml EP tube, where they were covered with an equal volume of 1640 medium (Basalmedia, shanghai, China) and incubated for 10 minutes. The intersection of the water phase and oil phase is where the micro-hydrogels would aggregate. For oil demulsification, extract 200 μl of liquid from the water-oil interface and add 20% of the 1H, 1H,2H,2H-perfluorooctanol (PFO). The micro-hydrogels were ultimately collected and counted after 15 minutes of incubation. FIG. 3B showed the image of micro-hydrogels. The counting method of micro-hydrogels was shown in Example 7.

In mouse experiments and human oocytes experiments, only a small quantity of micro-hydrogels was collected to meet ICSI requirements. Therefore, in order to avoid potential reproductive toxicity and achieve automated sperm selection procedures, the present invention used direct demulsification to reduce sperm exposure to chemicals. It was found that the microhydrogels sank at the bottom of the culture plate when the 1640 medium was added, due to demulsification and gravity (FIG. 3B). The sank hydrogels were transferred along with the aqueous phase (approximately 150 μL) entirely into the fertilization buffer (approximately 50 μL) containing alginate lyase (1 IU/ml) for hydrogel dissolution and sperm incubation, resulting in a final volume of the selected sperm solution of around 200 μL.

### EXAMPLE 7

### Efficiency of Droplet-To-Hydrogel Transformation and the Quantity of the Selected Sperm

It is well known that different studies and applications require varying sperm counts. For example, approximately 10-15 high-quality sperm need to be selected for ICSI, while more live sperm must be chosen for sperm cryopreservation or single-sperm in vitro culture or testing. It is essential to control the effectiveness of droplet-to-micro-hydrogel transformation and the quantity of selected sperm to meet the requirements of various prospective applications.

Following the foundational working principle of the BLASTO-chip, it is imperative for sperm to lower the pH of the droplet to below 6.8 during the incubation step. This reduction in pH facilitates the dissolution of calcium sulfate nanoparticles, enabling calcium ions to diffuse into the droplet and, ultimately, undergo cross-linking to form an alginate hydrogel.

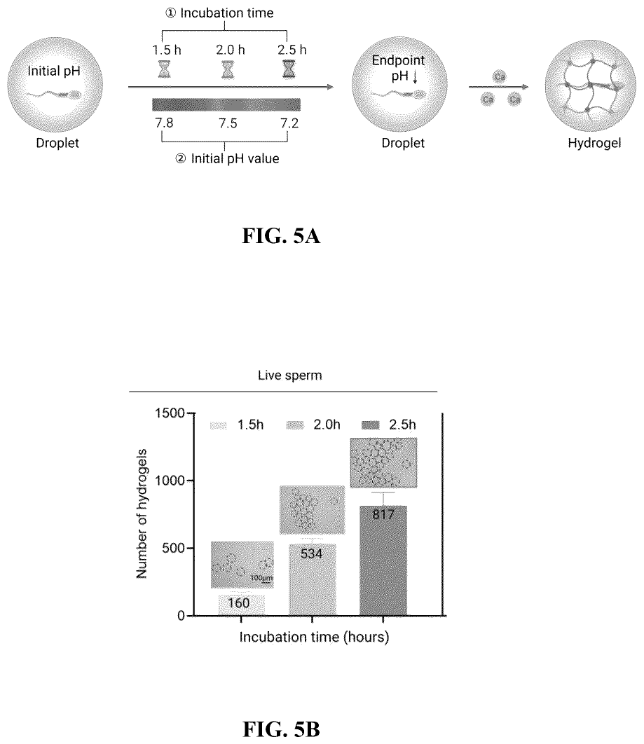

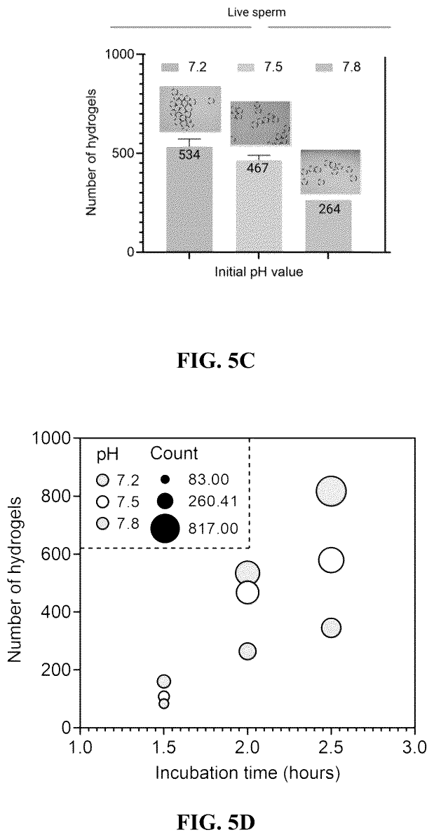

Consequently, the higher the initial pH of the droplet or the shorter the incubation duration, the more challenging the hydrogel formation becomes, requiring higher biochemical activity from the sperm (FIG. 5A). On the other hand, the initial pH of the droplet must also be conducive to the physiological state of the sperm. An excessively high or low pH can alter the normal metabolism of sperm, rendering the system incapable of selecting sperm under their physiological conditions.

Referring to FIG. 5A, a mixed samples having 30% live sperm were prepared and used to compare the number of hydrogels formed under different incubation times (1.5 h, 2.0 h and 2.5 h) or initial pH (7.2, 7.5 and 7.8). According to the experimental data shown in FIGS. 5B and 5D, the production of micro-hydrogels increased as the pH value decreased when the incubation time was kept constant. In contrast, when the initial pH value was fixed, the longer the incubation time, the more micro-hydrogels were produced (FIGS. 5C-5D). The detailed method for counting the number of micro-hydrogels were described in the Example 8.

### EXAMPLE 8

### Counting of Micro-Hydrogels

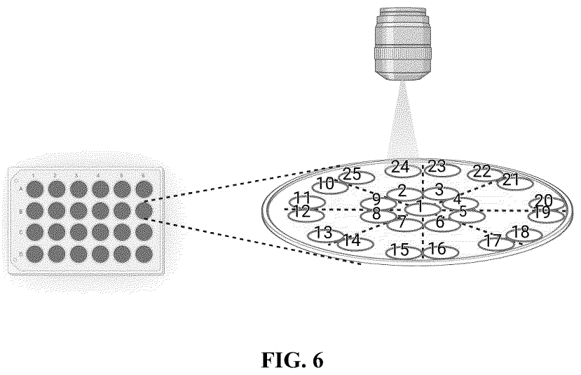

Turning to FIG. 6, the bottom of a culture plate with a diameter of 15.6 mm was evenly divided into 25 equal sections. Subsequently, the number of hydrogels in each area was calculated, and the average value was determined.

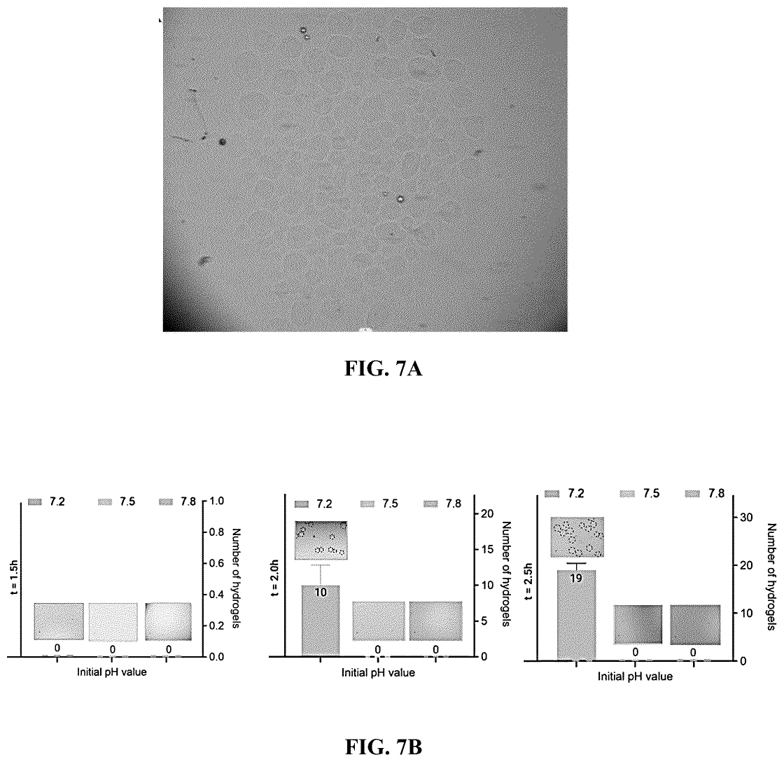

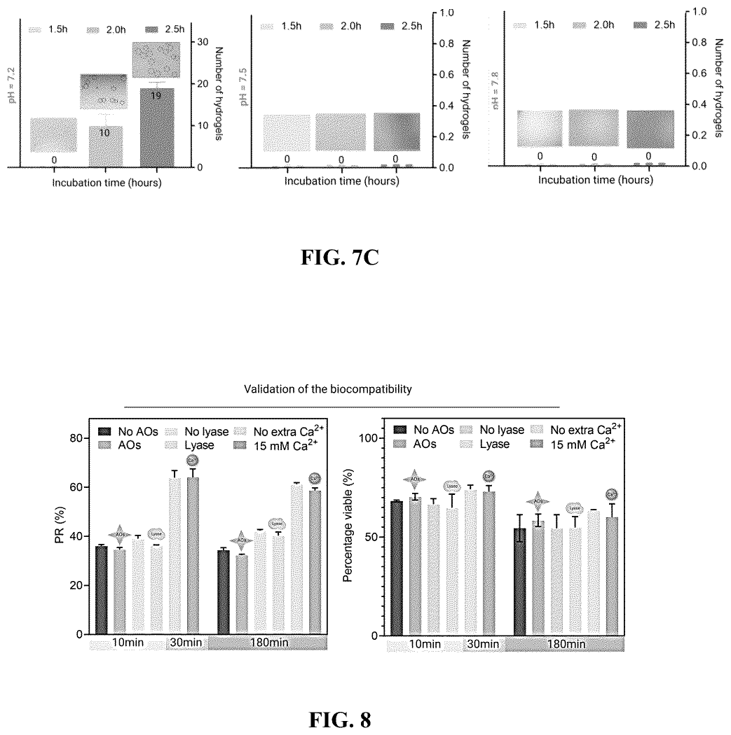

In parallel, the number of micro-hydrogels formed under the above conditions using samples with 0% live sperm was examined. FIGS. 7A-7C showed that under low initial pH (7.2) and long incubation time (2.0/2.5 h), a small fraction of droplets containing no sperm or dead sperm could form some irregularized micro-hydrogels, which was false positive event. For optimal specificity, users were advised to avoid conditions that could lead to false positives Taking into account factors such as selection efficiency and false selection rate, it was generally recommended to set the initial droplet pH at 7.5 and the incubation time at 2 hours.

Overall, the selection efficiency of the BLASTO-chip could be tuned by nearly 10-fold (from 83 to 817, as shown in FIG. 5D) simply by adjusting the initial droplet pH and the incubation time.

### EXAMPLE 9

Validation of the biocompatibility and reproductive toxicity of the BLASTO-chip system

The present invention utilizes a reversible ion crosslinking method to generate a sperm-encapsulating micro-hydrogels by using alginate and soluble calcium. This gentle crosslinking method is more suited for 3D cell culture. However, direct validation of its biocompatibility and reproductive toxicity is still extremely critical.

Therefore, the biocompatibility of all the components to which the sperm were exposed in the BLASTO-chip system was assessed, including 15 mM of calcium ions, 1 IU/mL alginate lyase and alginate oligosaccharides (AOs) (the degradation products of 1% alginates).

Turning to FIG. 8A, the results of verifying the biocompatibility of the BLASTO chip system are provided. The progressive motility (PR) (left side) and viability (right side) of sperm were measured before and after exposure to cell medium with or without alginate oligosaccharides (AOs), alginate lyase (1 IU/mL) and 15 mM Ca²⁺ for short (10 min/30 min) or prolonged (180 min) periods of time. Quantification of PR and viability were shown (all P>0.05) with unpaired two-tailed t test. Experimental results showed that regardless of whether sperm were exposed to those substances for short (10 min/30 min) or prolonged (180 min) periods of time, they did not significantly affect sperm' motility and viability compared to the control group (P>0.05).

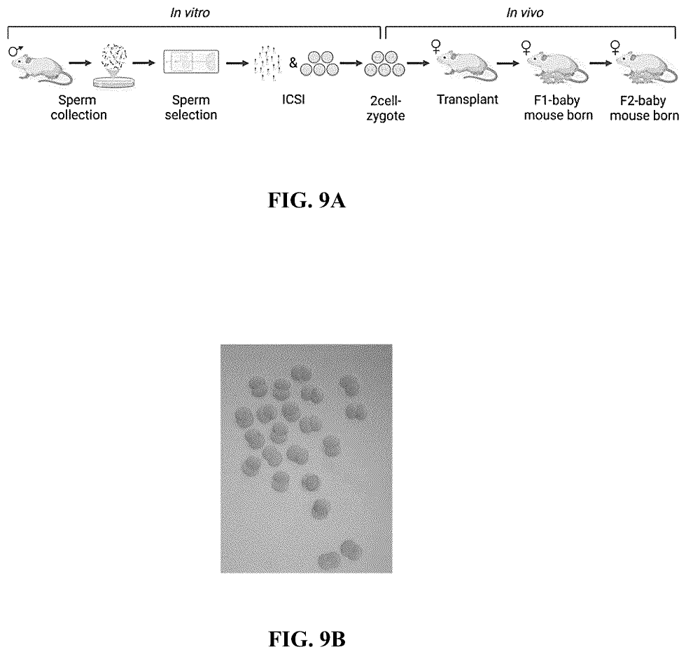

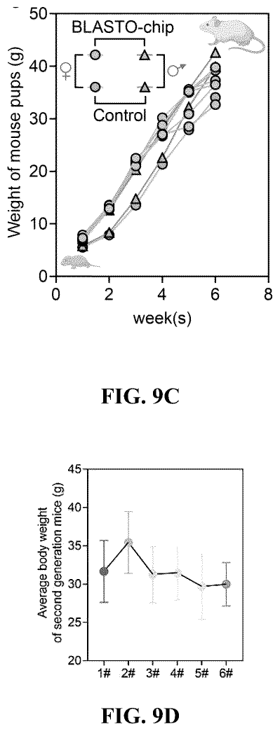

To further exhibit the system's reproductive non-toxicity of the BLASTO-chip, the present invention further carried out the ICSI-embryo transfer experiment on mice. The whole process was illustrated in FIG. 9A. Mouse sperm were selected using the BLASTO-chip, and after that, mouse oocytes were inseminated via ICSI. A total of 21 zygotes were transferred into the surrogate mice's oviducts once they had in vitro developed into 2-cell embryos in vitro (FIG. 9B). Seven newborn and 1-week-old first-generation (F1) mice were used and weighed weekly, and kept track of it to evaluate their growth and development. According to the results, there was no appreciable difference in growth when compared to mice born and raised in similar circumstances (P>0.05) (FIG. 9C). To further look into the potential long-term trans-generational effect on the offspring, when the F1 mice were 8 weeks old, they were naturally mated, and a total of 82 second generation (F2) mice were successfully born. Similarly, no significant difference was observed in the growth compared to the control group (P>0.05) (FIG. 9D).

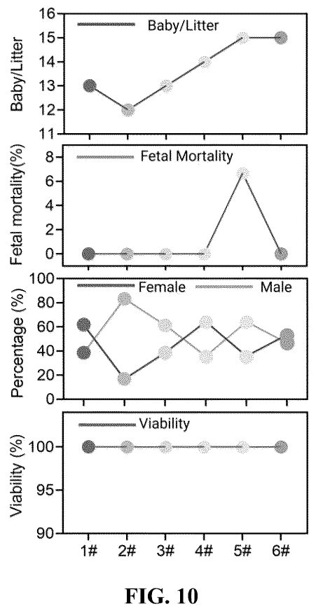

Additionally, the reproductive performance of each F1 mouse was assessed by analyzing the number of offspring per litter, fetal mortality, female-to-male ratio, and the viability of the corresponding F2 mice. The results in FIG. 10 showed that the reproductive performance of F1 mice was within the normal range.

The above data firmly demonstrates that the BLASTO-chip system of the present invention has no negative effects on sperm activity, reproduction performance and offspring health.

### EXAMPLE 10

Clinical relevance between sperm motility and the quantity of micro-hydrogels/selected sperm

In clinical practice, sperm progressive motility, quantitatively described by PR, is the most recognized parameter for assessing sperm quality. Therefore, the clinical relevance between sperm motility and the selection efficiency of the BLASTO-chip was investigated first.

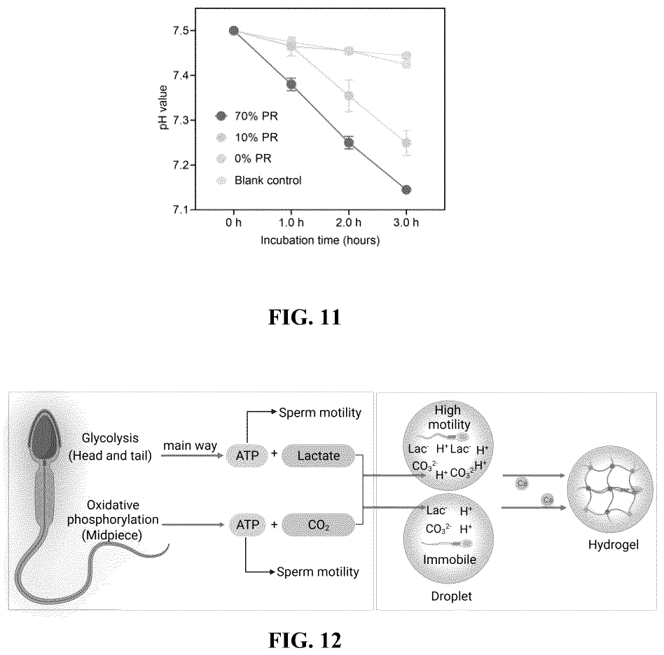

Anaerobic glycolysis, leading to lactate production, dominates the metabolism of human sperm, and the ATP it generates significantly influences sperm motility. Therefore, it can be reasonably assumed that sperm with higher motility metabolize at a faster rate, producing more acidic metabolic products, and lowering the surrounding environment's pH value more significantly. Experimental results in FIG. 11 have verified this assumption.

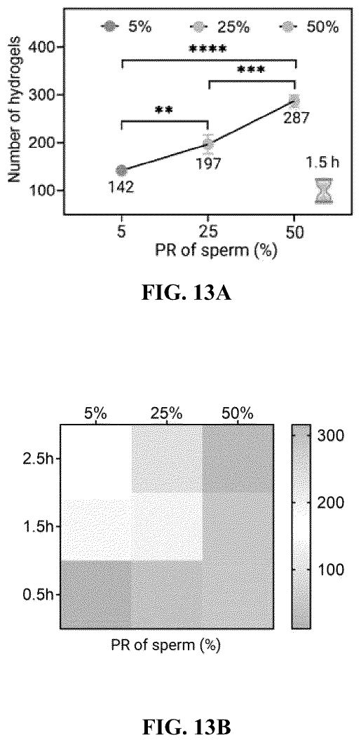

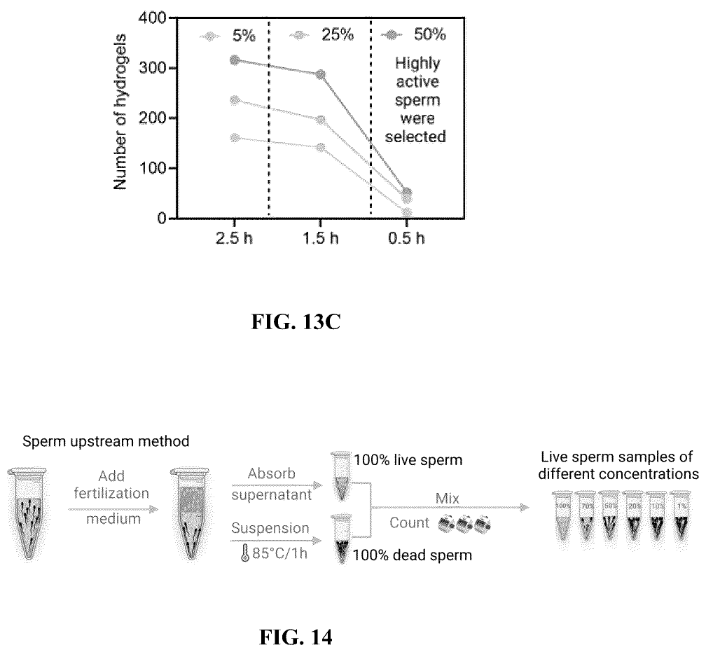

Given that the decrease in pH serves as the driving force for hydrogel formation, the present invention posits that the quantities of hydrogels/selected sperm, a measure of the sperm's ability to lower pH, will be positively correlated with the sperm's anaerobic glycolysis level and motility (FIG. 12). Utilizing BLASTO-chip technology, three semen samples with progressive motility rates (PR) of 5%, 25%, and 50% were processed by the CASA system. The term “PR” denotes the percentage of sperm that are moving forward in a straight line, or with only slight lateral or circular movement. The samples were treated with BLASTO-chip and as shown in FIGS. 13A-13B, the number of hydrogels/selected sperm increased with the PR percentages (5%, 25%, and 50%) or incubation time (t=0.5 h, 1.5 h, and 2.5 h), demonstrating that high-quality sperm samples could produce more hydrogels. Based on this, it is reasonable to deduce that the more active a sperm is, the lower the difficulty for it to form a hydrogel.

In FIG. 13C, it is evident that when the incubation time was reduced from 2.5 h to 0.5 h for the three aforementioned samples, only a small fraction of sperm could secrete sufficient acidic substances within the abbreviated incubation period, ultimately leading to their selection. This subset of sperm exhibited the highest biochemical activity.

The above findings showed the strong clinical relevance between sperm mobility and the biochemical activity of sperm. It was also worth noting that, based on the strong relevance mentioned above, the selection system held the potential for developing into a quantitative assay of sperm mobility and biochemical activity.

### EXAMPLE 11

The limit of selection of the BLASTO-chip toward human semen sample with ultra-low percentage of live sperm

For clinical application, it is vital to test whether the system can select live sperm from samples with low percentage of live sperm and determine its limit of selection toward such samples.

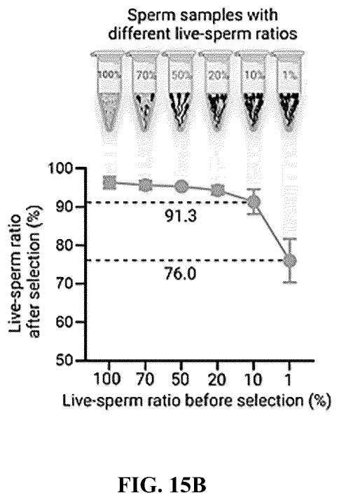

Turning to FIG. 14, a serial of mixed samples with the percentage of live sperm ranging from 1% to 100% were prepared. The BLASTO-chip was used to select sperm from those samples. After the selection process, sperm viability detection was conducted using eosanidine black staining for the selected sperm, and the percentage of live sperm was calculated (FIG. 15A). Experimental results in FIG. 15B showed that when the initial live-sperm percentage was above 10%, the average live-sperm percentage after selection all reached approximately 91.3%. Even for samples with only 1% live sperm, the live-sperm percentage could be elevated dramatically to 76% after selection, which was a 76-folds enhancement. The above data has firmly demonstrated the ultra-powerful capability of the BLASTO-chip in selectively isolating live sperm, significantly surpassing the number of non-viable sperm.

Numerous studies have indicated that sperm DNA damage is associated with male infertility, and the integrity of sperm DNA is crucial for conception and the development of healthy offspring. The sperm DNA fragmentation index (DFI), a crucial metric for assessing the quality of sperm, can represent the integrity of sperm DNA. Therefore, the DNA fragmentation index of selected sperm was assessed to determine the DFI values for three poor semen samples before and after selection.

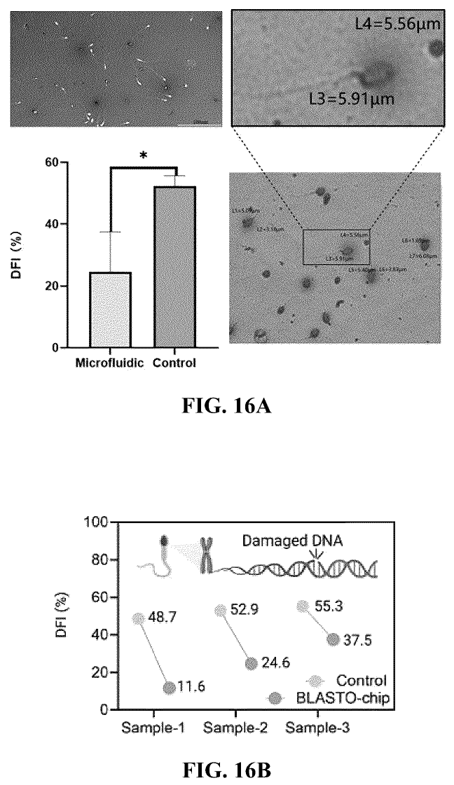

Compared with the untreated control group, the DFI values of all the tested samples dropped significantly after selection (FIGS. 16A-16B). These results demonstrated that the sperm selected by BLASTO-chip were not only alive, but also have high quality and potential of fertilization.

### EXAMPLE 12

Sperm selection from human semen samples with 10% live but 100% immobile sperm for ICSI

Patients with severe and absolute asthenozoospermia have endured a prolonged period of severely low fertilization rates, primarily due to the absence of biochemical-level sperm selection strategies. Traditional sperm selection methods based on physical parameters, such as morphology and kinematics, fail to capture the quality of sperm at the ‘biochemical’ level, and are unable to distinguish between viable and non-viable sperm. This ‘blind selection’ approach for patients with severe and absolute asthenozoospermia results in an exceptionally low fertilization rate.

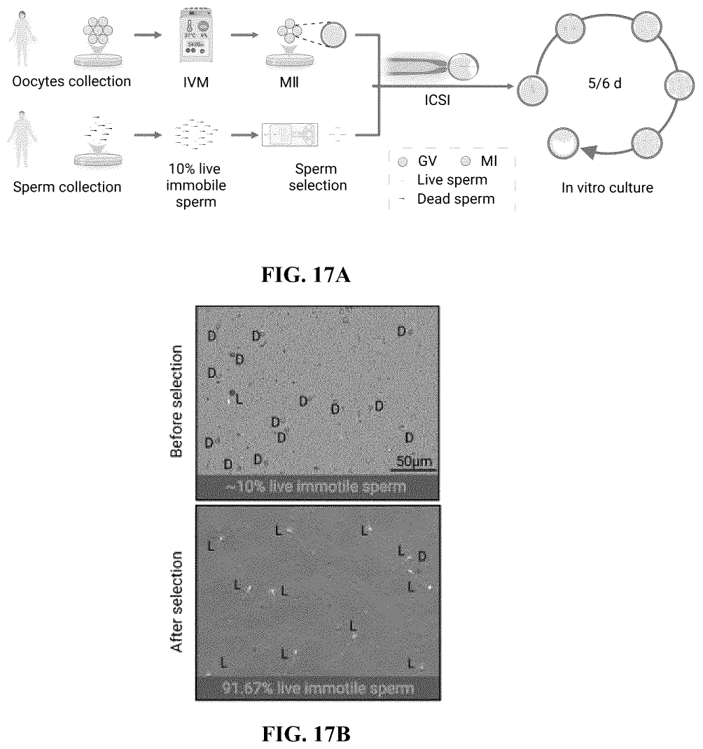

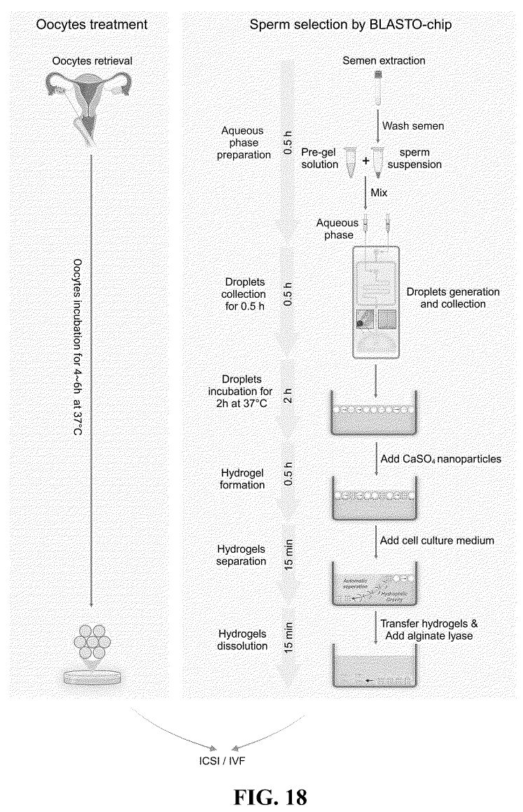

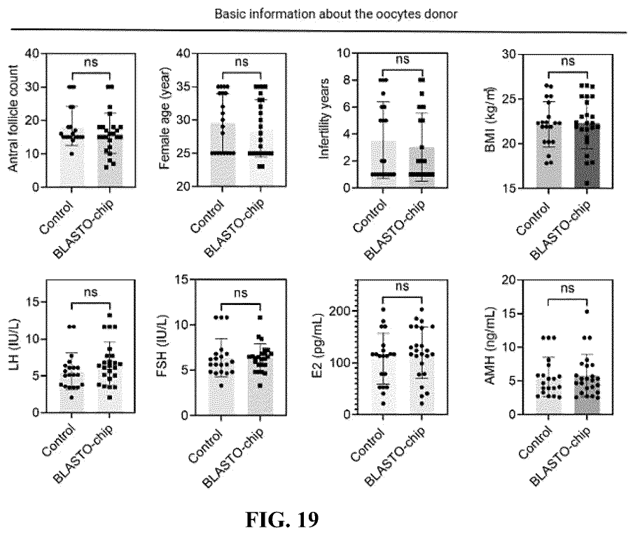

The present BLASTO-chip might be the solution eagerly awaited by them. To demonstrate this, semen samples containing 100% immobile but 10% alive sperm were prepared. Then, the BLASTO-chip was used for selection. FIG. 17B showed that the live-sperm percentage was elevated from 10% to 91.67% after selection. The selected sperm were further injected into human oocytes by ICSI (FIGS. 17A-17B, and FIG. 3A). For the parallel control group, experienced embryologists were invited to select sperm under a microscope and then perform ICSI. The whole selection timeframe of the BLASTO-chip was shown in FIG. 18 and it aligned with the clinical workflow. It was worth noting that there was no significant difference in the baseline data of the oocytes donors between the experimental and control groups (FIG. 19).

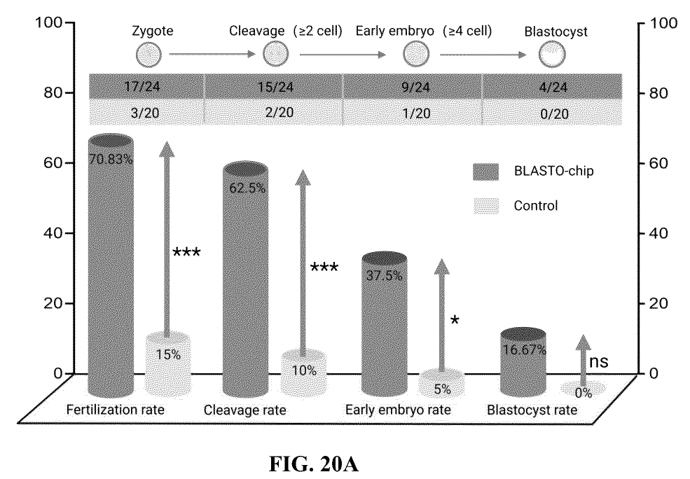

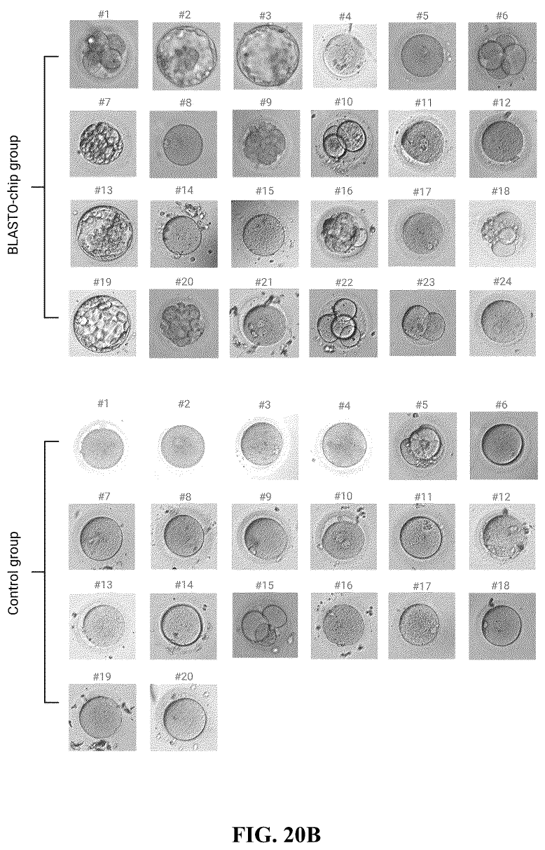

Finally, the fertilization rate, cleavage rate, early embryo rate and blastocyst rate between the two groups were compared to evaluate the clinical effectiveness of the BLASTO-chip system. As shown in FIGS. 20A-20B, for the experimental group, a total of 24 oocytes were tested, of which 17 oocytes were fertilized (70.83%), 15 zygotes were cleavaged (62.5%, 9 early embryos are formed (37.5%) and 4 embryos developed into blastocysts (16.67%). In contrast, in the control group (n=20), only 3 oocytes were fertilized (15%), 2 zygotes were cleavaged (10%), 1 early embryo are formed (5%), and no blastocyst was formed (0%). Table 6 showed the entire embryonic development process of the 4 blatocysts.

TABLE 6
Quantitative analysis of the fertilization rate, cleavage rate, early
embryo rate and blastocyst rate in BLASTO-chip and control groups

| | BLASTO-chip | | |
|---|---|---|---|
| | group | Control group | P value |
| No. of MII | 24 | 20 | |
| No. of fertilization rate | 70.83% (17/24) | 15% (3/20) | 0.0003 |
| (%) | | | |
| No. of cleavage rate | 62.5% (15/24) | 10% (2/20) | 0.0005 |
| (%) | | | |
| No. of early embro rate | 37.5% (9/24) | 5% (1/20) | 0.013 |
| (%) | | | |
| No. of blastocyst rate | 16.67% (4/24) | 0% (0/20) | 0.114 |
| (%) | | | |

### EXAMPLE 13

Verification of the relationship between pH changes caused by metabolically active sperm and sperm motility

To directly elucidate the relationship between pH changes and sperm motility, three experimental groups were set up-a high-motility sperm group (with a PR of approximately 70%), a weak-motility sperm group (with a PR of approximately 10%), and a dead sperm group (with a PR of 0%). The sperm was then incubated in a culture system without buffer at 37° C. After incubation of 1 h, 2 h and 3 h, their pH values were measured using pH electrode detector.

### EXAMPLE 14

### Quantification and Statistical Analysis

All analyses in this study were performed using GraphPad Prism 9 software. For multiple group comparisons, P values were calculated for multigroup comparison using a one-way ANOVA with Dunnett's multiple comparisons test. P values were calculated for two-group comparison using unpaired two-tailed t test. *P≤0.05, **P<0.01, ***P≤0.001, ****P<0.0001. Data were presented as the mean +s.d.

In summary, the BLASTO-chip system of the present invention is able to select biochemically active sperm with an accuracy of over 90%, and its selection efficiency could be flexibly tuned with the quantity of selected sperm ranging from 83 to 817. Notably, all the substances in the BLASTO-chip system will not pose any negative effect on the sperm viability or motility. It will not only provide great benefits to patients who have birth problems but also stimulate the development of further powerful tools in the area of reproduction.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### INDUSTRIAL APPLICABILITY

The present invention shows a dramatic therapeutic effect of the strategy toward a group of patients who have long suffered from ultra-low fertilization rate due to the extremely low mobility of sperm. Also, the formation of blastocyst further demonstrates the biosafety of the established BLASTO-chip system.

It will not only provide a powerful therapeutic tool for a large group of patients that suffer from low fertilization rate for ages but also lay a smart droplet-to-hydrogel microfluidic platform for development of more advanced sperm-selection technologies.

### Definition

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. The term “substantially coplanar” may refer to two surfaces within a few micrometers (μm) positioned along the same plane, for example, within 10 μm, within 5 μm, within 1 μm, or within 0.5 μm located along the same plane. When reference is made to “substantially” the same numerical value or characteristic, the term may refer to a value within ±10%, ±5%, ±1%, or ±0.5% of the average of the values.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

1. A microfluidic chip system for automatic separation of live sperm and subsequent formation of single-sperm-encapsulated microhydrogels, comprising:
a microfluidic chip, comprising:
at least one first channel for inflow of an aqueous phase, wherein the aqueous phase comprises sperm evenly dispersed in a solution;
at least one second channel for inflow of a flowing oil phase, wherein the at least one first channel and the at least one second channel are configured to create an interconnected cross-junction flow path to facilitate the mixing of the flowing oil phase and the aqueous phase, forming one or more single-sperm-encapsulated droplets;
an outlet for the one or more single-sperm-encapsulated droplets to flow out;
a culture plate connected to the outlet for droplet collection;
at least one pressure pump equipped with one or more syringes to establish connections between the at least one first channel and the at least one second channel to the one or more syringes, and
when a plurality of calcium sulfate nanoparticles is introduced into the culture plate and mixed with the one or more single-sperm-encapsulated droplets, droplet-to-hydrogel transformation is initiated and the single-sperm-encapsulated microhydrogels are formed,
wherein the microfluidic chip system selects biochemically active sperm with an accuracy of over 90%.

2. The microfluidic chip system of claim 1, wherein the flowing oil phase comprises:
a flowing oil comprising fluorinated oil, silicon oil, paraffin oil, mineral oil; and
a fluoro surfactant comprising perfluorooctanesulfonic acid (PFOS), perfluorooctanoic acid (PFOA), perfluorinated alkyl sulfonamido ethanols, fluorotelomer-based surfactants, perfluoropolyether-based surfactants.

3. The microfluidic chip system of claim 1, wherein the solution in the aqueous phase comprises an alginate salt comprising alginate, sodium alginate, potassium alginate, or a combination thereof.

4. The microfluidic chip system of claim 1, wherein the sperm have a concentration ranging from 1×10⁵ cells/mL to 1×10⁶ cells/mL.

5. The microfluidic chip system of claim 1, wherein the at least one first channel has a width ranging from 50-70 μm and a depth ranging from 80-100 μm, and the at least one second channel has a width ranging from 50-70 μm and a depth ranging from 80-100 μm.

6. The microfluidic chip system of claim 5, wherein the at least one first channel has a flow rate in a range of 1-10 μL/min, the at least one second channel has a flow rate in a range of 1-10 μL/min.

7. The method of claim 1, wherein the one or more single-sperm-encapsulated droplets have a uniformed size ranging from 60-120 μm and a pH value in a range of 7.0 to 8.0.

8. The method of claim 1, wherein the sperm contained in the single-sperm-encapsulated microhydrogels are viable, and the single-sperm-encapsulated microhydrogels have a pH value in a range of 3.0 to 7.0.

9. A method for improving the success rate of in vitro fertilization in a patient with asthenospermia, comprising the following steps:
processing semen samples and collecting sperm;
preparing a continuous phase and a dispersed phase, with each added to a syringe of the microfluidic chip system of claim 1, wherein the aqueous phase comprises sperm evenly dispersed in a solution;
mixing the continuous phase and the dispersed phase to form one or more single-sperm-encapsulated droplets, the one or more single-sperm-encapsulated droplets are collected on a culture plate;
adding calcium sulfate nanoparticles and cocultured them with the one or more single-sperm-encapsulated droplets for an incubation time to form single-sperm-encapsulated microhydrogels;
adding an aqueous culture medium into the culture plate to make the microhydrogels diffuse into the aqueous culture medium, while the droplets remain unchanged; and
adding alginate lyase to dissolve the microhydrogels and release selected sperm;
incubating the selected sperm in a fertilization medium for activation and subsequently injecting them into oocytes using microinjection,
wherein the method achieves a fertilization rate of at least 70% that is comparable to the fertilization rate obtained using sperm with normal quality.

10. The method of claim 9, wherein step of processing semen samples and collecting sperm further comprising subjecting the semen samples to density gradient centrifugation (DGC) or swim-up procedures to remove somatic cells and bacteria.

11. The method of claim 9, wherein the flowing oil phase comprises a flowing oil comprising fluorinated oil, silicon oil, paraffin oil, mineral oil; and a fluoro surfactant comprising perfluorooctanesulfonic acid (PFOS), perfluorooctanoic acid (PFOA), perfluorinated alkyl sulfonamido ethanols, fluorotelomer-based surfactants, pe sperma rfluoropolyether-based surfactants.

12. The method of claim 9, wherein the solution in the aqueous phase comprises an alginate salt comprising alginate, sodium alginate, potassium alginate, or a combination thereof.

13. The method of claim 9, wherein the sperm have a concentration ranging from 1×10⁵ cells/mL to 1×10⁶ cells/mL.

14. The method of claim 9, wherein the one or more single-sperm-encapsulated droplets have a uniformed size ranging from 60-120 μm and a pH value in a range of 7.0 to 8.0.

15. The method of claim 9, wherein the sperm contained in the single-sperm-encapsulated microhydrogels are viable, and the single-sperm-encapsulated microhydrogels have a pH value in a range of 3.0 to 7.0.

16. The method of claim 9, wherein the incubation time is in a range of 30 minutes to 3 hours.

17. The method of claim 9, wherein the selected sperm have a DNA fragmentation index of lower than 25%.

18. The method of claim 9, wherein the semen samples contain at least 1% live sperm with a progressive motility rate (PR) ranging from 0.1-100%.

19. The method of claim 18, when the PR of the sperm is less than 10%, the number of the selected sperm reaches at least 1×10⁴.

20. The method of claim 9, further comprising collecting oocytes from a subject, wherein the oocytes are in metaphase-II (MII) stage.
